(19) United States
(12) Patent Application Publication
(10) Pub. No.: US 2012/0116236 A1
(43) Pub. Date: May 10, 2012

(54) Hydration and blood flow adjusted glucose measurement

(76) Inventor: Joshua Noel Hogan, Los Altos, CA (US)

(21) Appl. No.: 13/373,081

(22) Filed: Nov. 3, 2011

Related U.S. Application Data
(60) Provisional application No. 61/456,286, filed on Nov. 4, 2010.

(51) Int. Cl. A61B 5/02; A61B 5/00

(52) U.S. Cl. 600/504; 600/300

(57) ABSTRACT

The invention provides a system and method for using OCT to determine hydration and blood flow variation adjusted glucose measurement in a target of interest. For some target, including a target of a living human, the invention provides for the measurement of tissue components, for determination of hydration level and blood flow variations of target, for non-invasive determination of glucose in target, and for hydration-adjusted and blood flow compensated glucose measurement in target.

In one embodiment, both the tissue components and glucose level are measured by OCT, and tables or indexes of hydration are used to determine hydration adjustment.

In alternate embodiments, the hydration index may be partially or fully “individualized,” created from data collected from monitoring an individual or groups of individuals with pre-selected characteristics (ex. same gender, weight, age, ethnicity, diabetes stage, species, etc.). Additional embodiments include blood flow variation monitoring and blood flow compensated glucose measurement.

### RELATED APPLICATIONS

This new utility application claims priority from U.S. provisional application 61/456,286 of the same title and by the same inventor, filed Nov. 4, 2010, the entirety of which is incorporated by reference as if fully set forth herein.

### GOVERNMENT FUNDING

N/A

### FIELD OF USE

The invention relates to measuring and monitoring composition of living tissue, and more particularly to the field of measuring glucose by means of techniques such as Optical Coherence Tomography (OCT).

### BACKGROUND

Hydration level changes and blood flow variations have the potential to interfere or confound measurement of glucose levels and hence reduce the accuracy of glucose measuring techniques including, but not limited to techniques based on Optical Coherence Tomography (OCT).

Dimensions of tissue components including, but not limited to: cells; thickness of layers, such as the dermis layer; vary with hydration and blood flow and thereby provide a method of measuring or monitoring hydration levels and blood flow variation in living entities, including humans.

Optical Coherence Tomography (OCT) can measure such tissue dimensions. Monitoring the dimensions of such tissue components by means of OCT provides a mechanism for monitoring hydration levels and blood flow variations, which can be used to compensate for the effect of hydration levels and blood flow variations on measured glucose levels, thereby increasing the accuracy of such glucose measurements, particularly suitable and economically viable in the case of OCT based glucose monitors.

### BRIEF SUMMARY OF THE INVENTION

The invention provides a system and method for using OCT to determine hydration and blood flow adjusted glucose measurement in a target of interest. For some target, including a target of a living human, the invention provides for the measurement of tissue components, for determination of hydration level and blood flow variations of a target, for non-invasive determination of glucose in target, and for hydration and blood flow adjusted glucose measurement in target.

In one embodiment, both the tissue components and glucose level are measured by OCT, and tables or indexes of hydration are used to determine hydration adjustment. In another embodiment, hydration levels are determined by other means, such as, for example, establishing daily weight baseline for a subject, or other known hydration measuring techniques. Glucose measurement in target tissue will be through a preferred method of OCT. The determination of hydration adjustment for the glucose measurement will be calculated by determining the hydration level of the target tissue, and introducing an adjustment factor accordingly.

It can be appreciated that hydration state bears upon other biometric readings, including glucose levels. When monitoring glucose in an individual, therefore, it is important to know reading validity across hydration states of that individual.

In other embodiments, the index of adjustment is generalized hydration adjustment tables. In alternate embodiments, the hydration index may be partially or fully “individualized,” created from data collected from monitoring an individual or groups of individuals with pre-selected characteristics (ex. same gender, weight, age, ethnicity, diabetes stage, etc.)

In other embodiments blood flow variations are monitored, measured and correlated to provide a blood flow compensated glucose level. While the invention is described in terms of human tissue characterization, it can be applied to veterinary applications, or any in vivo application where glucose monitoring is of interest.

### BRIEF DESCRIPTION OF THE DRAWINGS

FIG. 1 depicts OCT tissue analysis system, including glucose level measurement capability according to the present invention.

FIG. 2 is a flow chart setting forth the method according to a preferred embodiment.

### DETAILED DESCRIPTION OF A PREFERRED EMBODIMENT

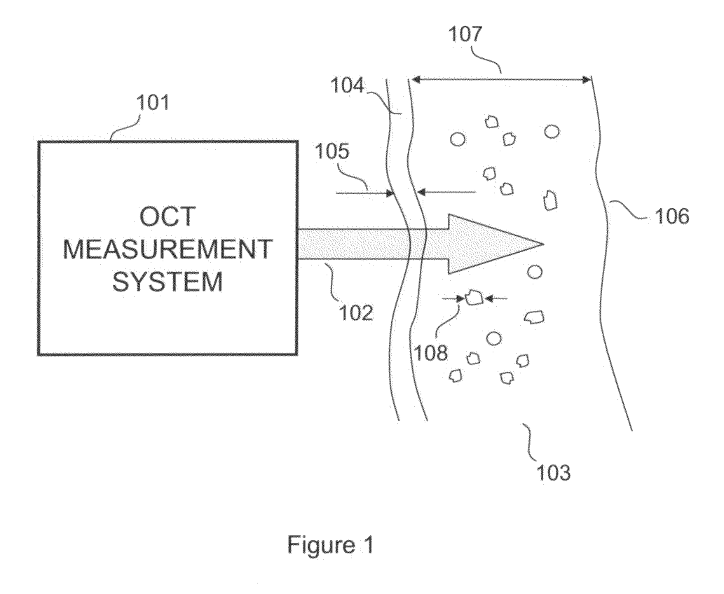

FIG. 1 depicts the manner in which OCT operates to obtain readings of tissue dimensions and glucose levels. Depicted in FIG. 1 are the following components:
101 OCT measurement system;
102 light noninvasively measuring characteristic of tissue 103 including dimensions of epidermis 104, such as average thickness, 105, the distance between the epidermis 104 and the dermis 106, indicated by the average distance 107, or average dimensions of structures such as cells 108.

The system as depicted is also useful in measuring glucose in tissue fluids, such as interstitial fluid, blood, etc. The operation of OCT for performing measurements in targets of interest is understood by a practitioner of average skill in the art. With respect to measurement of glucose in tissue with OCT, U.S. Pat. No. 7,526,329 entitled “Multiple Reference Non-invasive Analysis System” may be referred to. Also see “Specificity of noninvasive blood glucose sensing using optical coherence tomography technique: a pilot study, Phys. Med. Biol. 48 (2003) 1371-139, Kirill V Larin, Massoud Motamedi, Taras V Ashitkov and Rinat O Esenaliev.

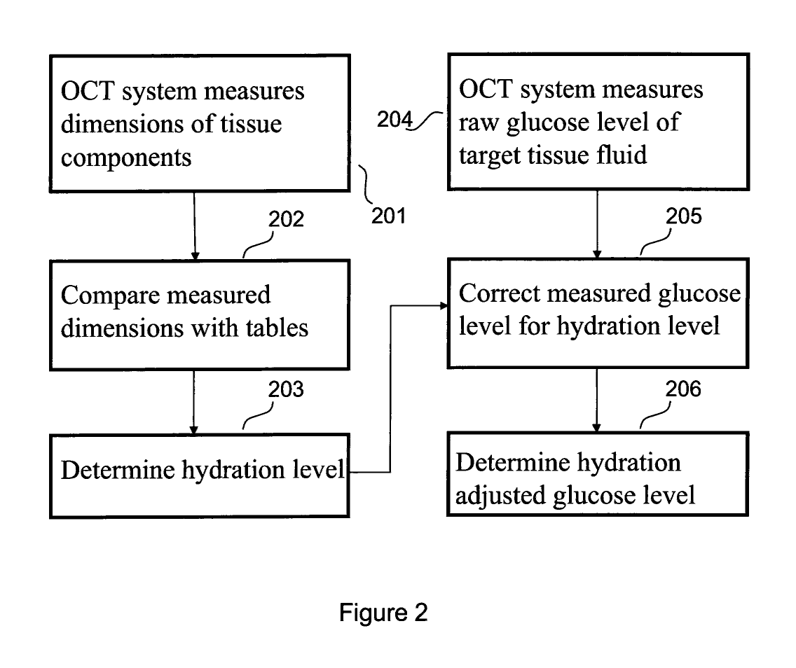

Referring to FIG. 2, the OCT system operating according to a preferred embodiment measures dimensions of tissue components performs the following steps:
Step 201 OCT system measures one or more predetermined dimensions of tissue component;
Step 202 Compare measured dimensions of tissue components with tables useful in determining hydration levels. It can be appreciated that a number of approaches to establishing hydration tables may be employed, including but not limited to conventional weight monitoring, urinalysis, fluid intake and outgo tracking, opto-thermal transient emission radiometry. Some relationship between component measurement variability and level of hydration will be reflected in the reference tables;
Step 203 Determine hydration level of target under analysis, by at least one comparison of the measured dimensions with appropriate indices/tables so as to determine hydration level;
Step 204 OCT system measures raw glucose level of target tissue fluid;
Step 205 Correct or adjust the measured glucose level of Step 204 for hydration level (as determined in step 203);
Step 206 Determine hydration adjusted glucose level and transmit/communicate or store said hydration adjusted glucose level.

As a practical matter, once tissue component dimensions are determined, and hydration level is established, then appropriate correction indices may be calculated for an individual (based on stored individual data) or a chart or table may be used to determine value correction.

For example, if a tissue measurement has a dimension X in hydration level H And has dimension X2 in hydration level H2, then some correction factor may be calculated or retrieved from a “look up” table with respect to accurately determining the amount of glucose present in the blood or other target sample. While the method discussed herein posits a determinable relationship between component dimension and hydration level, the inventive method include indirect indicators of hydration level. Moreover, there are a number of different approaches to generating an index of hydration-adapted glucose measurements. In some cases, the output of the hydration-adapted glucose measurement may be a number value. In others, the output may be indicated as a color shade, a bar value, or any other depiction that communicates glucose value.

In the case of an insulin dependent diabetic patient, for example, accurate glucose measurement is critically important to adjusting insulin dosages. To accurately use a non-invasive approach such as OCT it is important that variables such as hydration state be meaningfully addressed.

Blood flow in blood carrying vessels nearby the scan site, sufficiently close to the scan site to affect the scan, can likewise be monitored and useful data obtained. For example, blood flow in nearby blood carrying vessels can cause a variation in the location of the layer in the dermis, such as interfaces between the dermis, the papillary dermis and the reticular dermis layers.

By monitoring at least one dimension of a tissue component, storing the values of the measured dimension which becomes reference data, and correlating the measured dimension with reference data to determine a blood flow variation. This method provides a means to extract blood flow information, for example, pulse rate or heart rhythms.

This information may be further correlated with reference data to compensate for the effect of blood flow on glucose measurement and thereby improve the accuracy of glucose measurement.

Examples of measured dimension include, but are not limited, the diameter of a blood vessel, relative separation of layers with in the dermis, distance between a blood carrying vessel and a layer within the dermis.

The invention has veterinary applications, and in any case where accurate glucose monitoring data is desired.

We claim:

1. A method of monitoring and measuring hydration levels in living tissue, said method comprising:
monitoring (measuring) at least one dimension of at least one tissue component;
and
correlating said measured dimension with reference data to determine a hydration level.

2. The method of claim 1, wherein the dimension is the dimension of a tissue cell.

3. The method of claim 1, wherein the dimension is the dimension of a tissue layer.

4. The method of claim 3, wherein the tissue layer is the dermis layer.

5. A method of increasing accuracy of glucose measurement in living tissue, said method comprising:
monitoring (measuring) at least one dimension of at least one tissue component;
correlating said measured dimension with reference data to determine a hydration level;
and
using said determined hydration level to compensate measured glucose levels to improve accuracy of said measured glucose levels.

6. The method of claim 5, wherein the dimension is the dimension of a tissue cell.

7. The method of claim 5, wherein the dimension is the dimension of a tissue layer.

8. The method of claim 7, wherein the tissue layer is the dermis layer.

9. The method of claim 5, wherein measured glucose levels are measured using OCT based techniques.

10. A method of monitoring blood flow in living tissue, said method comprising:
monitoring (measuring) at least one dimension of at least one tissue component;
and
correlating said measured dimension with reference data to determine a blood flow variation.

11. The method of claim 10, wherein the dimension is the dimension of a blood carrying vessel.

12. The method of claim 10, wherein the dimension is the dimension of a tissue layer.

13. The method of claim 12, wherein the tissue layer is a dermis layer.

14. A method of increasing accuracy of glucose measurement in living tissue, said method comprising:
monitoring, where monitoring can include measuring, at least one dimension of at least one tissue component to produce a measured dimension, and storing values of said measured dimensions at pre determined intervals to create reference data;
correlating said measured dimension with reference data to determine a blood flow level;
and
using said determined blood flow level to compensate measured glucose levels to improve accuracy of said measured glucose levels.

15. The method of claim 14, wherein the dimension is the dimension of a blood carrying vessel.

16. The method of claim 14, wherein the dimension is the dimension of a tissue layer.

17. The method of claim 16, wherein the tissue layer is the dermis layer.

18. The method of claim 14, wherein measured glucose levels are measured using OCT based techniques.

19. The method as in claims 1, 5, 10 and 14, further including the step of distinguishing dimension changes and causes of said changes.

20. The method as in claim 19, wherein blood flow induced changes are identified.
